(19) United States
(12) Patent Application Publication
(10) Pub. No.: US 2026/0085389 A1
(43) Pub. Date: Mar. 26, 2026

(54) HIGH ENTROPY ALLOY FOR BIOIMPLANT APPLICATIONS

(71) Applicant: KING FAHD UNIVERSITY OF PETROLEUM AND MINERALS, Dhahran (SA)

(72) Inventors: Mohamed Abdrabou HUSSEIN, Dhahran (SA); Mohammed Abdul AZEEM, Dhahran (SA); Arumugam Madhan KUMAR, Dhahran (SA)

(73) Assignee: KING FAHD UNIVERSITY OF PETROLEUM AND MINERALS, Dhahran (SA)

(21) Appl. No.: 18/893,114

(22) Filed: Sep. 23, 2024

(51) Int. Cl. C22C 30/00; A61F 2/30; C22C 1/04

(52) U.S. Cl. CPC C22C 30/00; A61F 2/30; C22C 1/04

(57) ABSTRACT

A non-equiatomic high entropy alloy (HEA) includes titanium in an amount of 33 to 37 atomic percent, zirconium in an amount of 33 to 37 atomic percent, niobium in an amount of 18 to 22 atomic percent, tantalum in an amount of 3 to 7 atomic percent, and silver in an amount of 3 to 7 atomic percent, wherein atomic percent is based on the total atom count of the HEA. The elements titanium, zirconium, niobium, tantalum, and silver are randomly distributed within the HEA. A method of preparation thereof and its use thereof as a bioimplant with improved corrosion resistance, anti-bacterial properties, and reduced elastic modulus.

### STATEMENT REGARDING PRIOR DISCLOSURE BY THE INVENTORS

Aspects of the present disclosure are described in Hussein, M. A. et al., “Design and Development of Ti—Zr—Nb—Ta—Ag High Entropy Alloy for Biomedical Applications” published in Advanced Engineering Materials, which is incorporated herein by reference in its entirety.

### STATEMENT OF ACKNOWLEDGEMENT

Support provided by King Fahd University of Petroleum and Minerals, Saudi Arabia, through project ER221004 is gratefully acknowledged.

### BACKGROUND

### Technical Field

The present disclosure is directed to a non-equiatomic high entropy alloy (HEA) of 35Ti-35Zr-20Nb-5Ta-5Ag, methods of making the HEA, and uses thereof as a bioimplant with improved corrosion resistance, anti-bacterial properties, and reduced elastic modulus.

### Description of Related Art

The “background” description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Orthopedic implants have been developed to sustain or repair bone damage in humans caused by osteoarthritis, osteomas, arthritis, and other factors. A variety of metals and alloys, including titanium and titanium alloys, cobalt-chromium alloys, and stainless steel 316, have been used in biomedical application; however, not all metals and metal alloys meet implantation expectations, which include a low elastic modulus comparable to human bone. Furthermore, there have been concerns regarding the biocompatibility of certain metals, such as nickel (Ni), chromium (Cr), and cobalt (Co). The high specific strength, low Young's modulus, and biocompatibility of titanium and titanium alloys, compared to Co—Cr alloys and stainless steel 316, have increased interest in medical applications in the last two decades. In contrast to human bone, the titanium alloy, Ti64, has a relatively high elastic modulus (110 GPa). Titanium (Ti) alloys are classified into three classes based on their component phases (β, α+β, and α). The titanium alloy, Ti-6A1-4V (Ti64), is the most commonly utilized (α+β) type in medical applications [Bahl, S. et al., Comprehensive review on alloy design, processing, and performance of β titanium alloys as biomedical materials, Int. Mater. Rev. 2021, 66, 114]. T-6A1-4V may release aluminum (Al) and vanadium (V) ions into the surrounding human tissues; therefore, effort has been directed towards replacing Al and V with benign alloying elements such as zirconium (Zr), niobium (Nb), molybdenum (Mo), tantalum (Ta), tin (Sn), and silver (Ag). These alloying elements are known for their biocompatibility and ability to stabilize the β phase. As a result, recent research has focused on utilizing these biocompatible elements such as Ti—Nb—Ag [Hussein, M. A. et al., Ti-30Nb-3Ag alloy with improved corrosion resistance and antibacterial properties for orthopedic and dental application produced by mechanical alloying, J. Mech. Behav. Biomed., 2023, 142, 105851], Ti—Nb—Zr [Hussein, M. A., Synthesis, characterization, and surface analysis of near-P Ti20Nb20Zr alloy proceeded by powder metallurgy for biomedical applications, JOM, 2022, 74, 924], and Ti—Nb—Ta—Zr alloys [Li, B. Q. et al., Microstructure, mechanical property and corrosion behavior of porous Ti—Ta—Nb—Zr, Bioact. Mater., 2020, 5, 564]. Recently, the concept of high entropy alloys (HEAs) has been introduced [Cantor B., Multicomponent high-entropy cantor alloys, Prog. Mater. Sci., 2021, 120, 100754]. Unlike conventional alloys, high-entropy alloys (HEAs), which contain one and rarely two base elements, are of great research interest in materials science and engineering. HEAs comprise multiple principal elements, with the possible number of HEA compositions extending considerably more than conventional alloys. Properties of HEAs, such as their excellent specific strength, good mechanical performance at high temperatures, ductility and fracture toughness at cryogenic temperatures, superparamagnetic characteristics, and superconductivity, have been known [Ye et al., High-entropy alloy; challenges and prospects, 19(6) 2016 349]. HEAs with five or more element states provide an alloy with increased configurational entropy, which leads to high yield strength and corrosion protection [Hua, N. et al., Mechanical, corrosion, and wear properties of biomedical Ti—Zr—Nb—Ta—Mo high entropy alloys, J. Alloys Compd., 2021, 861, 157997]. Based on the concept of refractory high-temperature alloys, biomedical alloys have been developed.

A Ti-35Nb-5Ta-7Zr alloy for biomedical applications with a reduced Young's modulus of less than 10 CPa, comparable to that of human bones has been developed and studied [Li et al., A biomedical Ti-35Nb-5Ta-7Zr alloy fabricated by powder metallurgy, J. Material Engineering and Performance, 2019, 28, 5616]. In another study, Ti-20Zr-1ONb-4Ta (TZNT) alloy was tuned for antibacterial properties by introducing Ag, with broad-spectrum antibacterial properties, introduced on the surface of TZNT using a simple and efficient laser surface modification method [Xue et al. Antibacterial properties and cytocompatibility of Ti-20Zr-10Nb-4Ta alloy surface with Ag microparticles by laser treatment, Surface and Coatings Technology, 425, 2021, 127716]. Few patents, CN107488803 A, WO 2023/213385 A1. US 2002/0159914 A1, and WO 2016/080937 A1, cover high entropy multi-element biocompatible alloy for applications as an implant, a prosthesis, or as a thin film coating for an implant for use in medicine and veterinary surgery wherein the thin film coating being an alloyed thin film coating comprising multi elements.

Broadly, multi-element alloys are generally clinically good but cannot completely meet all performance requirements, and further application of metals like stainless steel is limited by pitting corrosion of the stainless steel, poor wear resistance of the titanium alloy, and moreover, the release of toxic metal ions of the cobalt-chromium-molybdenum alloy may cause microbial infections such as bacterial or fungal infections, when introduced into the body of a subject. Search for alloys for their biocompatibility, non-toxicity, and absence of allergic reactions is a continuous pursuit. One object of the present disclosure is to provide a non-equiatomic high entropy alloy that may circumvent the drawbacks of the present art.

### SUMMARY

In an exemplary embodiment, a non-equiatomic high entropy alloy (HEA) is described. The non-equiatomic high entropy includes titanium in an amount of 33 to 37 atomic percent, zirconium in an amount of 33 to 37 atomic percent, niobium in an amount of 18 to 22 atomic percent, tantalum in an amount of 3 to 7 atomic percent, and silver in an amount of 3 to 7 atomic percent. The atomic percent is based on the total atom count of the non-equiatomic high entropy alloy, and the titanium, zirconium, niobium, tantalum, and silver are randomly distributed within the non-equiatomic high entropy alloy.

In another exemplary embodiment, a process for making a non-equiatomic high entropy alloy is described. The process includes mixing a titanium powder, a zirconium powder, a niobium powder, a tantalum powder, and a silver powder to form a first mixture. Further, the method includes ball-milling the first mixture for 16 to 24 hours at a speed of 250 to 350 rpm, pressing the first mixture at a pressure of 500 to 750 MPa, and sintering the first mixture to a temperature of 1250 to 1350° C. to form the alloy.

In some embodiments, the method of ball-milling the first mixture is carried out with a bypass ratio of 5:1 to 15:1 in an inert environment.

In some embodiments, the process of pressing and sintering includes heating the first mixture to a temperature of 1250 to 1350° C. at a rate of 8 to 12° C./min to form the alloy.

In some embodiments, the process of sintering occurs for 1 to 3 hours.

In some embodiments, the alloy has a valence electron concentration of 3 to 5.

In some embodiments, the alloy has an atomic-size difference (6) of 4 to 6%.

In some embodiments, the alloy has an omega (Q) parameter of 10 to 11. The alloy has a major body-centered cubic 1 (bcc1) phase, a grain boundary body-centered cubic 2 (bcc2) phase, and an ultra-fine zirconium-rich equiaxed phase.

In some embodiments, the alloy has a grain size of 1 to 2 μm, a crystallite size of 3 to 4 nm, a melting temperature of 2150 to 2250 K, a density of 6.5 to 7.5 g/cm³, a Vickers microhardness of 3 to 6 GPa, and an elastic modulus of 80 to 120 GPa.

In some embodiments, a rate of antibacterial inhibition of the alloy in the presence of Bacillus subtilis is 90 to 94% greater compared a rate of antibacterial inhibition of a commercially pure titanium sample.

In some embodiments, a rate of antibacterial inhibition of the alloy in the presence of Escherichia coli is 83 to 90% greater compared a rate of antibacterial inhibition of a commercially pure titanium sample.

In some embodiments, the alloy has an impedance value of 70,000 to 300,000 Ω cm² in a stimulated body fluid medium and a phase angle of −75° to −85° in a stimulated body fluid medium.

In some embodiments, the reaction solution comprises a same volume of each of the first solution and the second solution.

In another exemplary embodiment, a bioimplant material comprising the non-equiatomic high entropy alloy is described.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a flowchart depicting a method of forming a non-equiatomic high entropy alloy (HEA), according to certain embodiments.

FIG. 2A depicts X-ray diffraction (XRD) patterns of a blended HEA mixture and a mechanically alloyed (MA) HEA for 20 hours, according to certain embodiments.

FIG. 2B depicts a XRD patterns of sintered HEA at 1300° C. and compacted at 550 MPa and 700 MPs, according to certain embodiments.

FIG. 3A shows a Scanning Electron Microscopy (SEM) image of blended HEA powder, according to certain embodiments.

FIG. 3B shows an SEM of MA HEA, according to certain embodiments.

FIG. 3C shows an SEM image of an HEA sintered sample at 1300° C. and compacted at 550 MPa, according to certain embodiments.

FIG. 3D shows SEM image of an HEA sintered sample at 1300° C. and compacted at 700 MPa, according to certain embodiments.

FIG. 3E shows an energy-dispersive X-ray spectroscopy (EDX) mapped image of the HEA sintered sample at 1300° C. and compacted at 550 MPa, according to certain embodiments.

FIG. 3F shows an EDX mapped image of the HEA sintered sample at 1300° C. and compacted at 700 MPa, according to certain embodiments.

FIGS. 4A-4E show Energy Dispersive X-ray (EDX) maps for the elements Ti, Zr, Nb, Ta, and Ag, respectively, of the MA HEA, according to certain embodiments.

FIGS. 4F-4J show EDX maps for the elements Ti, Zr, Nb, Ta, and Ag, respectively, of the sintered HEA sample at 1300° C. and compacted at 550 MPa, according to certain embodiments.

FIGS. 4K-40 show EDX maps for the elements Ti, Zr, Nb, Ta, and Ag, of the sintered HEA sample at 1300° C. and compacted at 700 MPa, according to certain embodiments.

FIG. 5A shows the load-displacement curve for the HEA sintered sample at 1300° C. and compacted at 550 MPa (a) and 700 MPa (b), according to certain embodiments.

FIG. 5B shows the elastic modulus of the HEA compared to commercial alloys, according to certain embodiments.

FIG. 6A depicts a potentiodynamic polarization (PDP) plot showing in vitro corrosion results of the HEA sintered sample at 1300° C. and compacted at 550 MPa and 700 MPa and a Ti64 alloy tested in simulated body fluid (SBF), according to certain embodiments.

FIG. 6B is a Nyquist plot of the HEA sintered sample at 1300° C. and compacted at 550 MPa and 700 MPa and Ti64 alloy tested in SBF, according to certain embodiments.

FIG. 6C is a Bode plot of the HEA sintered sample at 1300° C. and compacted at 550 MPa and 700 MPa and the Ti64 alloy tested in SBF, according to certain embodiments.

### DETAILED DESCRIPTION

In the following description, it is understood that other embodiments may be utilized, and structural and operational changes may be made without departure from the scope of the present embodiments disclosed herein.

When describing the present disclosure, the terms used are to be construed in accordance with the following definitions, unless a context dictates otherwise. Embodiments of the present disclosure will now be described more fully hereinafter with reference to the accompanying drawings wherever applicable, in which some, but not all embodiments of the disclosure are shown. In the drawings, like reference numerals designate identical or corresponding parts throughout the several views. Further, as used herein, the words “a,” “an,” and the like generally carry a meaning of “one or more,” unless stated otherwise. Within the description of this disclosure, where a numerical limit or range is stated, the endpoints are included unless stated otherwise. Also, all values and subranges within a numerical limit or range are specifically included as if explicitly written out.

Furthermore, the terms “approximately,” “approximate,” “about,” and similar terms generally refer to ranges that include the identified value within a margin of 20%, 10%, or preferably 5%, and any values therebetween.

As used herein, the terms ball-milling, sintering, body-centered cubic (bcc) unit cell, and face-centered cubic (fcc) unit cell refer to conventional definitions known in the literature.

As used herein, “Vickers microhardness, HV”, measures the hardness of a sample, and the unit of hardness given by the test is known as the Vickers Pyramid Number (HV).

As used herein, “Young's modulus” (abbreviated as E) is an intrinsic material parameter that describes the relationship between the unitless percent elongation of a material, that is, strain (ε), and the force applied per area, that is, stress (σ).

As used herein, “non-equiatomic” refers to an alloy, including a high entropy alloy, comprising five or more principal alloying elements, in proportions that are not equal or near equal,

Unless otherwise noted, the present disclosure is intended to include all isotopes of the samples used herein.

Aspects of the present disclosure are directed to a non-equiatomic 35Ti-35Zr-20Nb-5Ta-5Ag high entropy alloy (HEA), also referred to as a Ti—Zr—Nb—Ta—Ag HEA, HEA, HEA alloy, and alloy, for use in a bioimplant. Mechanical alloying is used to synthesize the HEA from elemental powders, which are then compacted and sintered at 1300° C. The HEA was evaluated for its chemical and mechanical properties, and the HEA of the present disclosure demonstrated improved corrosion resistance and reduced elastic modulus compared to the commercially available Ti6A14V alloy, making it a strong candidate for bioimplant applications.

A non-equiatomic high entropy alloy (HEA) or alloy is described. The alloy includes titanium in an amount of 33 to 37 atomic percent (at. %), zirconium in an amount of 33 to 37 atomic percent, niobium in an amount of 18 to 22 atomic percent, tantalum in an amount of 3 to 7 atomic percent, and silver in an amount of 3 to 7 atomic percent, wherein atomic percent is based on the total atom count of the non-equiatomic HEA. In a specific embodiment, the alloy includes titanium in an amount of 33 to 37 at. %, preferably 34 to 36 at. %, and yet more preferably about 35 at. %, zirconium in an amount of 33 to 37 at. %, preferably 34 to 36 at. %, and yet more preferably about 35 at. %, niobium in an amount of 18 to 22 at. %, preferably 19 to 21 at. %, and yet more preferably about 20 at. %, tantalum in an amount of 3 to 7 at. %, preferably 4 to 6 at. %, and more preferably about 5 at. %, and silver in an amount of 3 to 7 at. %, preferably 4 to 6 at. %, and more preferably about 5 at. %, wherein atomic percent is based on the total atom count of the non-equiatomic HEA. The elements titanium, zirconium, niobium, tantalum, and silver are randomly distributed within the non-equiatomic HEA.

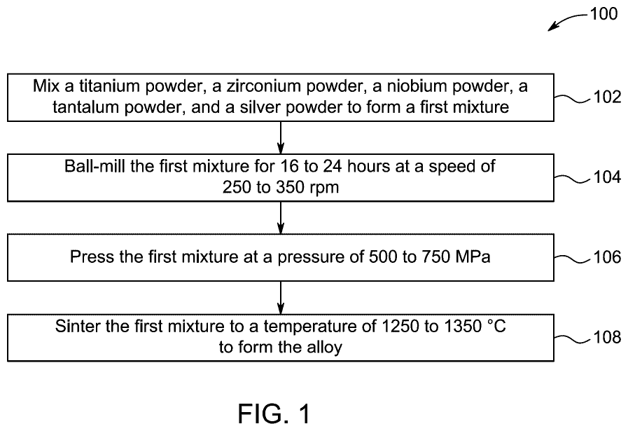

Referring to FIG. 1, a method 100 of preparing the non-equiatomic high entropy alloy is described. The order in which the method 100 is described is not intended to be construed as a limitation, and any number of the described method steps may be combined in any order to implement the method 100. Additionally, individual steps may be removed or skipped from method 100 without departing from the spirit and scope of the present disclosure.

At step 102, the method 100 includes mixing a titanium powder, a zirconium powder, a niobium powder, a tantalum powder, and a silver powder to form a first mixture. Elemental powders of Ti, Zr, Nb, Ta, and Zr, according to the atomic percentage ranges as defined earlier, are physically mixed and/or blended to form the first mixture. The titanium powder, zirconium powder, niobium powder, tantalum powder, and/or silver powder may be pre-blended individually before being physically mixed and/or blended to form the first mixture. The physical mixing and/or blending may occur by any means known in the art. Each of the elemental powders has a purity of at least 99%, preferably at least 99.5%, more preferably at least 99.9%. The first mixture, formed by blending the elemental powders, has a crystallite size in the range of 25 to 30 nm, preferably 26 to 29 nm, more preferably 27 and 28 nm, and yet more preferably about 27.59 nm. The first mixture, formed blending the elemental powders, has an average particle size in the range of about 12.2±5 μm.

At step 104, the method 100 includes ball-milling the first mixture for 16 to 24 hours, preferably 17 to 23 hours, preferably 18 to 22 hours, more preferably 19 to 21 hours, and yet more preferably about 20 hours, at a speed of 250 to 350 rpm, preferably 260 to 340 rpm, preferably 270 to 330 rpm, preferably 280 to 320 rpm, more preferably 290 to 310 rpm, and yet more preferably about 300 rpm. During the ball-milling process, the first mixture is loaded into the sample vial or grinding jar of a grinder or grinding machine, such as a planetary ball mill. The sample vial is filled with grinding media, such as grinding balls configured to rotate about its axis during milling. The milling or grinding mechanism is provided by the movement of the sample vial and the grinding media. In one embodiment, the sample vial is made of tungsten carbide and is partially filled with tungsten carbide balls at a ball-to-powder ratio of 10:1 (bypass ratio). In some embodiments, the sample vial and grinding media are made of any material known in the art. The grinding media may be metallic or non-metallic. Metallic grinding media may be made of carbon steel, chrome steel, stainless steel, steel shot, tungsten carbide, or any other acceptable alloy or metal. Non-metallic grinding media may be made of alumina, ceramic (steatite), glass, flint, nylon, silicon carbide, silicon nitride, tungsten carbide, synthetic polymers, and any other non-metallic material known in the art. The ball-to-powder ratio (by weight) is in a range of 5:1 to 15:1, preferably 7:1 to 13:1, more preferably 8:1 to 12:1. In a preferred embodiment, the ball-to-powder ratio is 10:1. In an embodiment, the ball diameters are 5-20 mm, preferably 10-20 mm, and more preferably about 10 mm. In one embodiment, tungsten carbide balls are used in the ball milling process, and the sample vial is also made of tungsten carbide. This process is known as mechanical alloying.

Mechanical alloying processes may include the use of a process control agent, such as stearic acid and/or methanol, to minimize cold welding of the elemental powder particles and to prevent the powders from sticking to the grinding media and the vial wall. In the present disclosure, the addition of a process control agent is not used to avoid contamination of the elemental powders.

The first mixture is agitated by ball-milling or any other grinding techniques known in the art at room temperature and under an inert atmosphere (i.e., in the presence of an inert gas such as argon or nitrogen, preferably argon) at 250 to 350 rpm, preferably 270-320 rpm, more preferably 290-310 rpm, and yet more preferably 300 rpm. The first mixture is ground for 16-24 h, 18-22 h, more preferably 20 hours. After mechanical alloying, the first mixture has an average particle size of 22.9±9 μm, and the particles are agglomerated.

At step 106, the method 100 includes pressing the first mixture at a pressure of 500 to 750 MPa. The first mixture is compacted in a graphite die (about 20 mm³) by applying a suitable pressure of about 500 to 750 MPa, preferably 525 to 725 MPa, and more preferably 550 to 700 MPa. In an embodiment, the first mixture is compacted in a graphite die at a pressure of about 550 MPa, also referred to as HEA550 and HEA550 MPa. In another embodiment, the first mixture is compacted in a graphite die at a pressure of about 700 MPa, also referred to as HEA700 and HEA700 MPa.

At step 108, the method 100 includes sintering the first mixture to a temperature of 1250 to 1350° C. to form the alloy. The compacted/pressed first mixture is further subjected to sintering by placing the first mixture in a spark plasma sintering (SPS) chamber or furnace. In some embodiments, spacers may be used. The sintering is carried out under an argon atmosphere to a temperature of 1250 to 1350° C., preferably 1260 to 1340° C., preferably 1270 to 1330° C., preferably 1280 to 1320° C., more preferably 1290 to 1310° C., and yet more preferably about 1300° C., at a heating rate of 8 to 12° C./min, preferably 9 to 11° C./min, and more preferably about 10° C./min, for 1 to 3 hours, preferably 1.5 to 2.5 hours, and more preferably about 2 hours to form the alloy.

The alloy's microstructures, revealed by scanning electron microscopy, show the occurrence of dual-phase body-centered cubic (bcc) structures and an ultra-fine zirconium-rich equiaxed phase. The bcc structures are a major body-centered cubic 1 (bcc1) phase and a grain boundary body-centered cubic 2 (bcc2) phase. In an embodiment, the bcc1 phase includes about 35-40 at. % titanium, preferably about 38 at. % titanium, 30-35 at. % zirconium, preferably about 31 at. % zirconium, 20-25 at. % niobium, preferably about 22 at. % niobium, 1-10 at. % tantalum, preferably about 5 at. % tantalum, and 1-5 at. % silver, preferably about 3 at. % silver. In an embodiment, the bcc2 phase includes about 25-30 at. % titanium, preferably about 26 at. % titanium, 10-15 at. % zirconium, preferably about 13 at. % zirconium, 25-35 at. % niobium, preferably about 29 at. % niobium, 1-10 at. % tantalum, preferably about 8.8 at. % tantalum and 10-30 at. % silver, preferably about 21 at. % silver. In an embodiment, the equiaxed dark phase includes about 75-85 at. % zirconium, preferably about 82 at. % zirconium, 10-15 at. % titanium, preferably about 13 at. % titanium, 1-10 at. % tantalum, preferably about 2 at. % tantalum, and 1-3 at. % silver, preferably about 2.3 at. % silver. The equiaxed dark phase is substantially free of niobium.

In some embodiments, the alloy has a melting temperature of 2150 to 2250 K, preferably 2175 to 2225 K, more preferably 2190 to 2000 K, and yet more preferably about 2193.65 K. In some embodiments, the alloy has a density of 6.5 to 7.5 g/cm³, preferably 6.6 to 7.4 g/cm³, preferably 6.7 to 7.3 g/cm³, more preferably 6.8 to 7 g/cm³, and more preferably about 6.89 g/cm³. In some embodiments, the alloy has a valence electron concentration of 3 to 5, preferably 3.5 to 4.5, more preferably 3.8 to 4.3, and yet more preferably about 4.1. In some embodiments, the alloy has an atomic-size difference (6) of 4 to 6%, preferably 4.5 to 5.5%, more preferably 4.8 to 5.2%, and yet more preferably about 5.003%. In some embodiments, the alloy has an omega (Q) parameter of 10 to 11, preferably 10.1 to 10.5, more preferably 10.2 to 10.3, and yet more preferably about 10.221. In some embodiments, the alloy has a crystallite size of about 3 to 5 nm, preferably 3.2 to 4 nm, more preferably 3.4 to 3.8 nm, and yet more preferably about 3.6 nm.

In some embodiments, the alloy has a grain size of 1 to 2 μm, preferably 1.5 to 1.8 μm. The grain size is dependent on the pressure applied during compaction. In an embodiment, when a pressure of about 550 MPa is applied to the first mixture, the sintered alloy or the alloy has a grain size of about 1.5 μm. In another embodiment, when a pressure of about 700 MPa is applied to the first mixture, the sintered alloy or the alloy has a grain size of about 1.8 μm.

In some embodiments, the alloy has an impedance value of 70,000 to 300,000 Ω cm² in a stimulated body fluid (SBF) medium, preferably 90,000 to 280,000 Ω cm², preferably 110,000 to 260,000 Ω cm², preferably 130,000 to 240,000 Ω cm², preferably 150,000 to 220,000 Ω cm², or preferably 170,000 to 200,000 Ω cm². In some embodiments, the alloy has a phase angle of −75° to −85°, preferably −76° to −84°, preferably −77° to −83°, preferably −78° to −82°, more preferably −79 to −81, and yet more preferably about −80° in the SBF medium.

Vickers hardness tests were performed on the HEA nanoalloys. In some embodiments, the Vickers hardness tests comply with at least one of the ASTM E92 Standard Test Method for Vickers Hardness of Metallic Materials and the ASTM E384 Standard Test Method for Microindentation Hardness of Materials, which are incorporated herein by reference in their entireties. In some embodiments, the alloy has a microhardness or Vickers hardness of 3-6 GPa, preferably 3.3-5.5 GPa, and more preferably 3.4 to 5.4 GPa. In some embodiments, the alloy is compacted at a pressure of 550 MPa and has a Vickers hardness of 3.47±0.1 GPa. In some embodiments, the alloy is compacted at a pressure of 700 MPA and has a Vickers hardness of about 5.35 GPa.

In some embodiments, wherein the alloy possesses high fracture stress, having Young's modulus values of 80-120 GPa, preferably 83 to 115 GPa, or preferably 90-110 GPa. These modulus values are lower than the values for Ti64 alloys, biomedical grades of Co-based alloys, and stainless steels. The alloys of the present disclosure are corrosion-resistant, biocompatible, and have higher wear resistance and durability, therefore making them suitable for various biomedical and dental implant applications. As used herein, the terms “tensile modulus,” “Young's modulus,” “elastic modulus,” and “modulus” refer to a measure of stiffness of a material defined as the ratio of the stress (force per unit area) along an axis to the strain (ratio of deformation over initial length). Modulus of elasticity can be measured by nanoindentation or compression tests, such as the ASTM E9-09 Standard Test Methods of Compression Testing of Metallic Materials at Room Temperature (incorporated herein by reference in its entirety).

The alloy of the present disclosure may be used in biomedical, orthopedic, and dental implants. Such parts include, but are not limited to, pins, rods, screws, plates, nails, wires, bars, posts, films, coatings, and the like. Examples of biomedical implants incorporating the Ti alloy include, but are not limited to, artificial pacemakers, coronary stents, contraceptive implants (intrauterine devices or IUD), cochlear implants, mechanical heart valves, expandable rib cages, spinal fusion cages, maxi-facial prosthetics, and the like. A non-exhaustive list of orthopedic implants includes Austin-Moore prosthesis, Baksi's prosthesis, Buttress plate, Charnley prosthesis, condylar blade plate, Ender's nail, Grosse-Kempf nail, Harrington rod, Hartshill rectangle, Insall Burstein prosthesis, interlocking nail, Kirchner wire, Kunscher nail, Luque rod, Moore's pin, Neers' prosthesis, rush nail, Smith Peterson nail, Smith Peterson nail with McLaughlin's plate, Seidel nail, Souter's prosthesis, Steffee plate, Steinmann pin, Swanson prosthesis, Talwalkar nail, Thompson prosthesis, total hip replacement system, hip resurfacing system, total knee replacement system, finger or toe replacement system, shoulder/ankle/elbow replacement system, femoral nail, tibial nail, and the like. Dental implants that can incorporate the alloy described herein include, but are not limited to, root-form implants, Ramus-frame implants, transosseous implants, blade-form implants, and the like. The alloy may also be used in surgical instruments or devices. Examples of such devices include, but are not limited to, surgical forceps, retractors, suture instruments, surgical tweezers, scissors, needle and micro needle holders, dental scalers, dental elevators, dental drills, endodontic files and reamers, Lasik eye surgery equipment, laser electrodes, vena cava clips, and the like.

The alloy of the present disclosure, when implemented in a bioimplant, may efficiently kill bacteria and/or inhibit bacterial infection, thus preventing implant failure. The alloy is effective against gram-positive and gram-negative bacteria. In a specific embodiment, the rate of antibacterial inhibition of the alloy in the presence of Bacillus subtilis is 90 to 94%, preferably 91 to 93%, or preferably about 92% greater compared to the rate of antibacterial inhibition of a commercially pure titanium sample. In yet another embodiment, the rate of antibacterial inhibition of the alloy in the presence of Escherichia coli is 83 to 90%, preferably 84 to 89%, preferably 85 to 88%, or preferably 86 to 87% greater compared to the rate of antibacterial inhibition of a commercially pure titanium sample.

The examples below are intended to further illustrate protocols for preparing, characterizing, and using the non-equiatomic high-entropy alloy and for performing the method described above and are not intended to limit the scope of the claim.

### EXAMPLES

The following examples describe and demonstrate non-equiatomic high-entropy alloy (HEA) preparations and use. The examples are provided solely for illustration and are not to be construed as limitations of the present disclosure, as many variations are possible without departing from the spirit and scope of the present disclosure.

### Example 1: Materials

High purity elemental titanium (Ti), zirconium (Zr), niobium (Nb), and tantalum (Ta) was purchased from Alfa Aesar. Silver (Ag) was purchased from US Research Nanomaterials, Inc powders.

### Example 2: Synthesis of High Entropy Alloy (HEA)

The 35Ti-35Zr-20Nb-5Ta-5Ag HEA powders were synthesized using the mechanical alloying technique on a planetary Micro Mill PULVERISETTE 7 premium line machine using high-purity Ti, Zr, Nb, Ta, and Ag powders. The HEA powder mixture was loaded into tungsten carbide (WC) vials with a diameter of 10 mm in diameter, with WC grinding balls in the ball-to-powder ratio (BPR) of 10:1 under high-purity argon. The blended HEA powder was ball milled for 20 hours at a rotational speed of 300 rpm without a process control agent to avoid contaminations. The mechanically alloyed HEA powders were compacted in a uniaxial press, automatic hydraulic press machine (TMAX-ZYP, TMAXCN, China) in a 20 mm die at 550 MPa and 700 MPa, and then sintered at 1300° C. in a tube furnace with a heating rate of 10° C./min for 2 hours. under argon atmosphere. The HEA sintered samples compacted at 550 MPa and 700 MPa were labeled as HEA550 and HEA700, respectively.

### Example 3: Characterization Study Using X-Ray Diffraction (XRD)

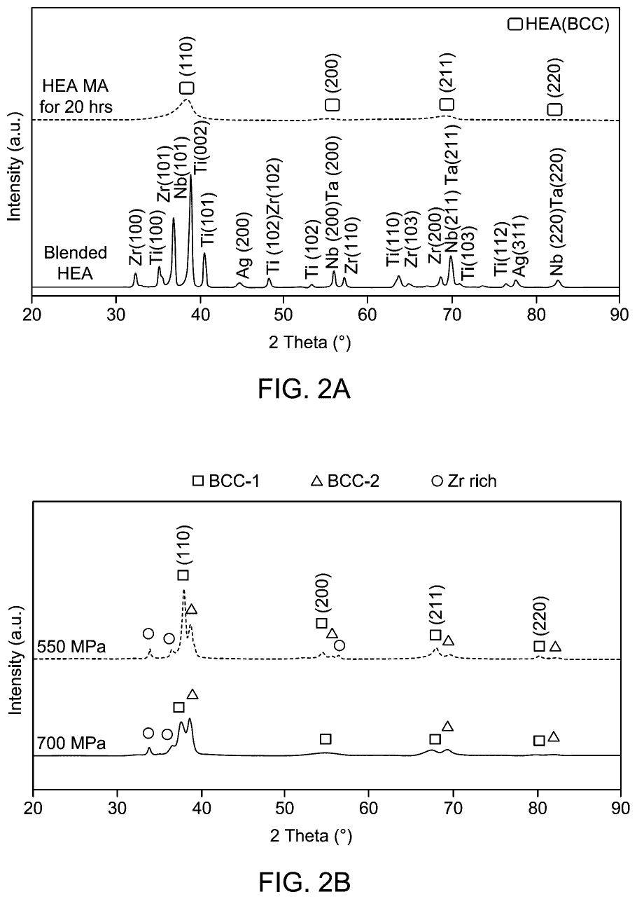

Physical properties, such as phase composition, crystal structure, and orientation of powder, solid, and liquid samples, were analyzed by XRD. The samples were analyzed using XRD (2°/min from 20 to 90°), and the phases of mechanically alloyed and sintered HEA samples were investigated. Full width at half maximum (FWHM) of XRD profiles was used to characterize different material properties and surface integrity features. The XRD patterns of the blended HEA mixture, mechanically alloyed HEA for 20 hours, and sintered HEA are shown in FIG. 2A. The presence of crystalline peaks of all constitution elements of HEA (Ti, Zr, Nb, Ta, and Ag) with no peaks related to contaminations is shown in FIG. 2A. After mechanical alloying (MA), the crystalline peaks of the constitution elements of HEA were replaced with broad peaks with reduced intensity compared to the blended alloy powders (FIG. 2B). An increased FWHM indicates a reduction in crystallite size, and the broadening of MA peaks indicates the refinement in crystallite size and high lattice strain [Xiang T. et al., Phase-tunable equiatomic and non-equiatomic Ti—Zr—Nb—Ta high-entropy alloys with ultrahigh strength for metallic biomaterials, Mater. Sci. Technol., 2022, 117, 196; Suryanarayana, C. et al., Synthesis of nanocrystalline ZnSe by mechanical alloying, Journal of Materials Research, 2023, 38, 4261, both of which are incorporated herein by reference in their entireties]. Based on the FWHM and 20 position, the Scherrer equation was used to determine the crystallite size. The crystallite size of mechanical alloyed HEA for 20 hours was measured to be 3.6 nm compared to 27.59 nm for the as-blended HEA powder. The diffraction peaks increasingly expanded and the intensity of the peaks decreased after the ball milling process. The size mismatch effect, additional grain boundary fraction, and additional dislocation density contribute to high lattice strain [Zhang, K. B. et al., Nanocrystalline CoCrFeNiCuAl high-entropy solid solution synthesized by mechanical alloying, J. Alloys Compd., 2009, 485, L31, which is incorporated herein by reference in its entirety]. A single solid solution phase with a bcc crystal structure indexed with crystallographic planes of (110), (200), (211), and (220) was formed in mechanically alloyed HEA samples as seen in FIG. 2A. The XRD pattern of sintered HEA at 1300° C., compacted at 550 MPa and 700 MPa is shown in FIG. 2B. The diffraction peaks of the bulk HEA samples are sharper and more intense than those of the ball-milled HEA powders due to the stress release and grain coarsening that occurs during the sintering process [Xiang, T. et al, Dual phase equal-atomic NbTaTiZr high-entropy alloy with ultra-fine grain and excellent mechanical properties fabricated by spark plasma sintering, Mater. Sci. Technol., 2021, 90, 150, which is incorporated herein by reference in its entirety]. The sintered HEA alloy showed a crystalline peak indexed for bcc-1 and bcc-2 phases and a Zr-rich phase, as shown in FIG. 2B, with nanocrystalline sizes of 17.57 nm, 13.98 nm, and 29.85 nm, respectively. The higher intensity of bcc-1 peaks in comparison to other phases indicates that bcc-1 is the dominant fraction and other phases exist in lesser amounts.

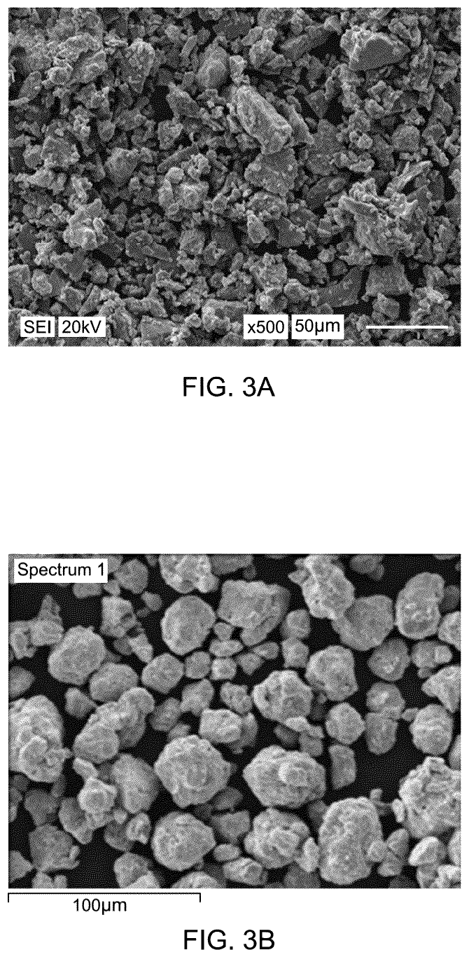

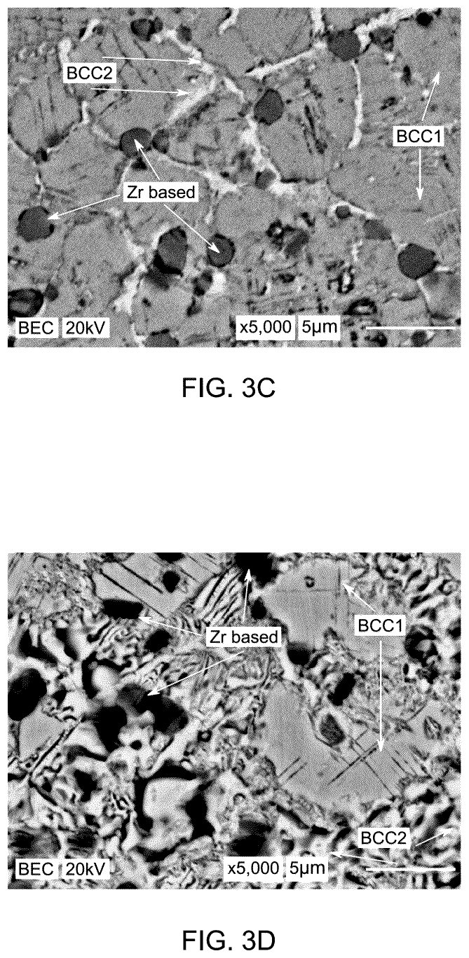

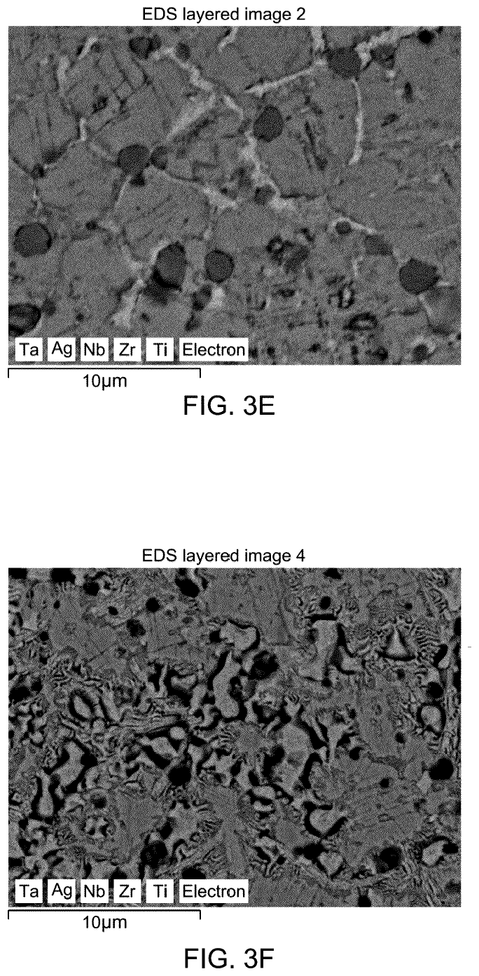

The microstructure and elemental analysis were done using scanning electron microscopy (SEM, JEOL) equipped with energy dispersive spectroscopy. Following sintering, samples were prepared for microscopic examination using standard procedures. Initially, SiC water-based grinding wheels with grit sizes ranging from 240 to 1200 were used. This was followed by fine polishing with Al₂O₃ fine powder to achieve a mirror-like surface. Following that, the samples were etched for 5 seconds in 10% HF to reveal the phases and grain structure. The mechanically alloyed powders, as shown in FIG. 3B, are agglomerated with an average particle size of 22.9±9 μm and cold-welded, according to an SEM micrograph, while the blended powders had sizes in the range of 12.2±5 μm as in FIG. 3A due to the high surface energy of the fine particles.

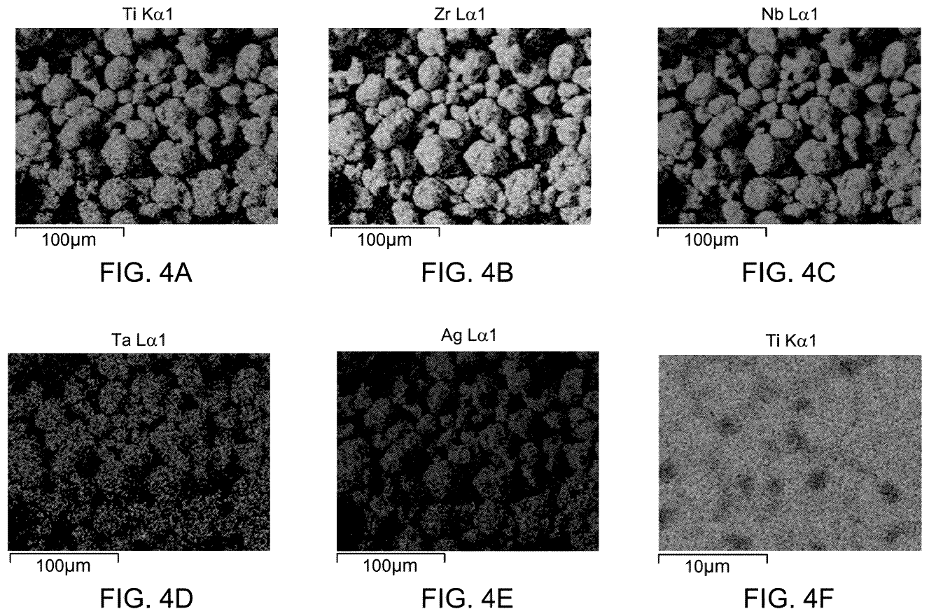

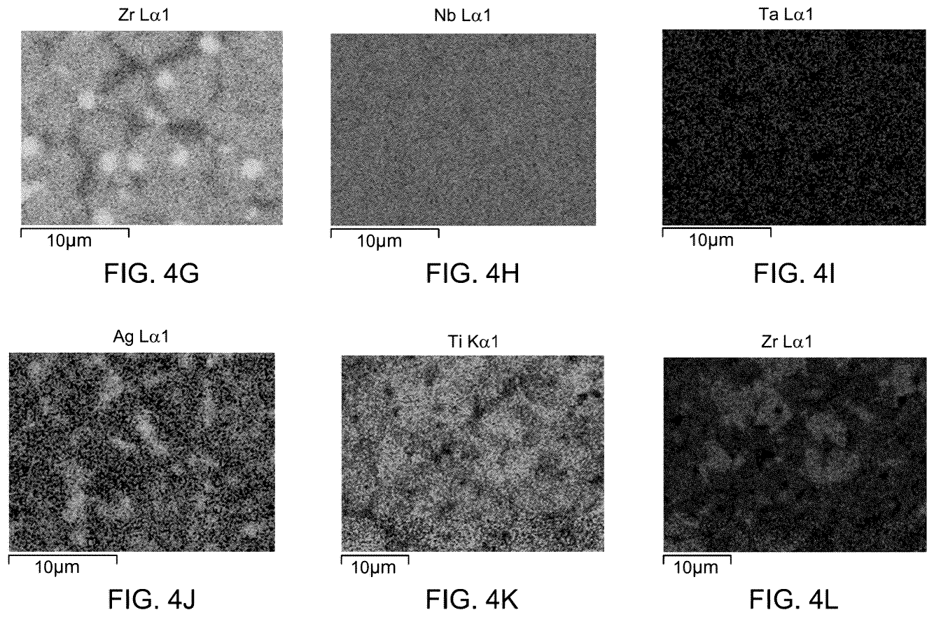

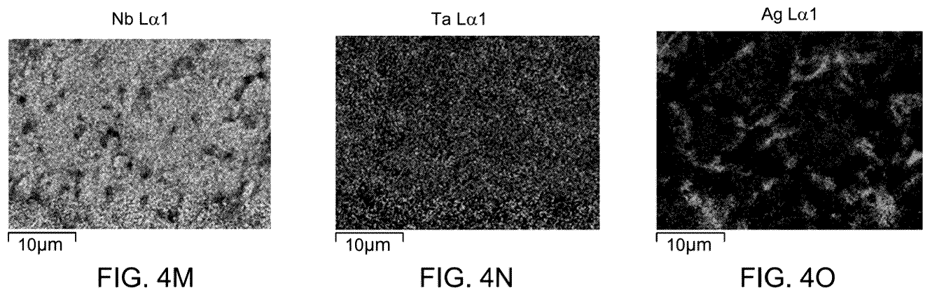

Energy Dispersive X-ray (EDX) microanalysis is a technique of elemental analysis associated with electron microscopy based on the generation of characteristic X-rays that reveal the presence of elements in the samples. EDX mapping revealed the homogeneous distribution of the alloying elements after ball milling for 20 hours, as shown in FIGS. 4A-4E. The backscattered electron (BSE) images and EDX (elemental) mapped images of sintered HEA are depicted in FIGS. 3C-3F. BSE images reveal different phases in the sintered HEA samples. The observed phases in XRD analysis results were further verified in the SEM analysis. The SEM analysis reveals the formation of two main phases with bcc crystal structure, matrix bcc 1 and grain boundary phase with bcc 2, along with ultra-fine equiaxed phase. The bcc 1 and bcc 2 phases include all the constitution elements of HEA, indicating the formation of multicomponent Ti—Zr—Nb—Ta—Ag HEA. The elemental analysis of the matrix bcc1 phase showed Ti 38 at. %, Zr 31 at. %, Nb 22 at. %, Ta 5 at. %, and Ag 3 at. %, and the bcc2 phase showed Ti 26 at. %, Zr 13 at. %, Nb 29 at. %, Ta 8.8 at. %, and Ag 21 at. %. The equiaxed dark phase showed Zr-rich phases with Zr 82 at. %, Ti 13 at. %, Ag 2.3 at. %, and Ta 2 at. %. Light and heavy elements in back scattered electron (BSE) mode exhibit distinct manifestations. This alludes to the centralized distribution of heavy items in the bright contrast region and light elements in the dark contrast region. Thus, it can be deduced that Nb and Ta elements are concentrated in the bright contrast region while Ti and Zr elements are centrally distributed in the dark contrast region. The size of the equiaxed Zr-rich phase and the bcc1 phase in the HEA550 sample were measured as 1.5±0.3 μm and 5.6±1.3 μm, respectively (FIG. 3C). On the other hand, the sizes of equiaxed Zr-rich phase and matrix bec1 phase in HEA700 were measured as 1.8±0.4 μm and 6±1.5 μm, respectively (FIG. 3D).

### Example 4: Phase Characterization of High Entropy Alloy (HEA)

Thermodynamic characteristics for the formation of HEA were used to determine the chemical composition of the HEA [Zhang, Y. et al., Solid-solution phase formation rules for multi-component alloys, Adv. Eng. Mater., 2008, 10, 534, which is incorporated herein by reference in its entirety]. Equations. 1-5 were used to estimate thermodynamic parameters of the Ti—Zr—Nb—Ta—Ag HEA system to determine the formation of solid solution phases.

$$Δ {S}_{mix} = - R {\sum }_{i = 1}^{n} {c}_{i} ln {C}_{i} \qquad ( Eq . 1 )$$

$$Δ {H}_{mix} = 4 {\sum }_{i = 1 , i \neq j}^{n} Δ Hij CiCj \qquad ( Eq . 2 )$$

$$δ = 100 \sqrt{{\sum }_{i = 1}^{n} ci {( 1 - \frac{ri}{\underline{r}} )}^{2}} where \underline{r} = {\sum }_{i = 1}^{n} Ciri \qquad ( Eq . 3 )$$

$$Ω = {T}_{m} Δ {S}_{mix} / \vert Δ {H}_{mix} \vert \qquad ( Eq . 4 )$$

$$VEC = \sum ci VECi \qquad ( Eq . 5 )$$

The formation of stable solid-solution phases is contingent upon the conditions δ≤6.6% and Ω≥1.1, and a ΔHₘᵢₓ value in the range of greater than or equal to 2.1 and less than or equal to 3.6 kJ/mol may also be beneficial [Yang, X. and Zhang, Y., Prediction of high-entropy stabilized solid-solution in multi-component alloys, Mater. Chem. Phys., 2012, 132, 233, which is incorporated herein by reference in its entirety]. Ω is the high-entropy phase formation factor, 6 is the atomic-size difference, ΔHₘᵢₓ is the heat of mixing, and VEC is the valence electron concentration (VEC). Table 1 lists the characteristics of the Ti—Zr—Nb—Ta—Ag HEA component elements, mixing enthalpy of binary alloys found in HEA, and calculated thermodynamic parameters. The calculated values of δ and Ω for the 35Ti-35Zr-20Nb-5Ta-5Ag HEA were 5% and 10.22, respectively. These values were found to be approximately in agreement with the criterion of the formation of a solid-solution phase showing that the Ti—Zr—Nb—Ta—Ag HEA system has potential to produce a solid solution phase. From a broad thermodynamic perspective, it is anticipated that high entropy effects will promote the development of solid-solution phases [Yeh, J-W., Alloy design strategies and future trends in high-entropy alloys, JOM, 2013, 65, 1759, which is incorporated herein by reference in its entirety]. When the ΔHₘᵢₓ between unlike atomic pairs is low in multicomponent systems, the entropy component (ΔSₘᵢₓ) of Gibbs free energy (ΔGₘᵢₓ) can potentially compete with the ΔHₘᵢₓ and promote the formation of a simple solid-solution phase [Guo, N. N. et al., Microstructure and mechanical properties of refractory MoNbHfZrTi high-entropy alloy, Mater. Des., 2015, 81, 87, which is incorporated herein by reference in its entirety]. Conversely, a high ΔHₘᵢₓ may encourage the formation of more than two phases, including ordered intermetallic [Fazakas, É. et al., Experimental and theoretical study of Ti20Zr20Hf20Nb20X20 (X=V or Cr) refractory high-entropy alloys, Int. J. Refract. Met. Hard Mater., 2014, 47, 131, which is incorporated herein by reference in its entirety]. The emergence of HEAs prompts new questions about mechanisms underlying their phase stability and methods for screening compositions for the formation of a single solid-solution phase. Valence Electron Concentration (VEC) can be used as an indicator in the HEA system to determine the phase stability of fcc or bcc solid solutions. The fcc solid solution phases were found to be more stable at a higher VEC (≥8), while the bcc solid solution phases were stabilized at lower VEC (<6.87) [Guo, S. et al., Effect of valence electron concentration on stability of fcc or bcc phase in high entropy alloys, J. Appl. Phys., 2011, 109, 103505, which is incorporated herein by reference in its entirety]. The present 35Ti-35Zr-20Nb-5Ta-5Ag HEA has a VEC value of 4.1, which meets the upper boundary of the bcc case as described. According to the parameters, the 35Ti-35Zr-20Nb-5Ta-5Ag HEA produces a single solid-solution with a bcc crystal structure, which was obtained after mechanical alloying for 20 hours (see FIG. 2A); however, after the sintering process, dual bcc phases were obtained with an ultrafine Zr-based phase, as shown in FIG. 2B. A solitary bcc solid solution phase may not form due to a phase separation phenomenon, and a miscibility interval may exist in the bcc phase [Xiang T. et al., Phase-tunable equiatomic and non-equiatomic Ti—Zr—Nb—Ta high-entropy alloys with ultrahigh strength for metallic biomaterials, Mater. Sci. Technol., 2022, 117, 196, which is incorporated herein by reference in its entirety]. The atomic pairings of Ti—Nb (+2), Ti—Ta (+1), Zr—Nb (+4), Zr—Ta (+3), Nb—Ag (+16), and Ta—Ag (+15) have a positive enthalpy of mixing [Wang, S-P. et al., TiZrNbTaMo high-entropy alloy designed for orthopedic implants: As-cast microstructure and mechanical properties, 2017, 73, 80, which is incorporated herein by reference in its entirety].

TABLE 1
Values used in the calculation of HEA alloying elements and
the calculated empirical parameters for TiZrNbTaAg HEA

| | | | | | | | TiZrNbTaAg |
|---|---|---|---|---|---|---|---|
| Element | | Ti | Zr | Nb | Ta | Ag | HEA |
| Atomic number | | 22 | 40 | 41 | 73 | 47 | — |
| Atomic weight | | 47.88 | 91.24 | 92.91 | 180.94 | 107.868 | — |
| Lattice structure, room | | HCP | HCP | BBC | BBC | FCC | — |
| temperature | | | | | | | |
| Lattice structure, high | | BBC | BBC | BBC | BBC | FCC | — |
| temperature | | | | | | | |
| Lattice parameter, Å | | 3.276 | 3.582 | 3.301 | 3.303 | 4.09 | — |
| Melting temperature, K | | 1933 | 2125 | 2741 | 3269 | 1234 | 2193.65 |
| Atomic radius, nm | | 0.1462 | 0.1603 | 0.1429 | 0.143 | 0.144 | — |
| Density, g/cm³ | | 4.51 | 6.51 | 8.57 | 16.7 | 10.49 | 6.89 |
| Mixing enthalpy | Ti | — | 0 | 2 | 1 | −2 | 2.42 |
| ΔHₘᵢₓ (kJ/mol) | Zr | | — | 4 | 3 | −20 | |
| | Nb | | | — | 0 | 16 | |
| | Ta | | | | — | 15 | |
| | Ag | | | | | — | |

| | | | | | | |
|---|---|---|---|---|---|---|
| Vickers microhardness, | 0.97 | 0.903 | 1.32 | 0.873 | 0.251 | 0.975 |
| HV (GPa) | | | | | | |
| Mixing entropy | — | — | — | — | — | 11.276 |
| ΔSₘᵢₓ (J/mol K) | | | | | | |
| δ | — | — | — | — | — | 5.003 |
| Ω | — | — | — | — | — | 10.221 |
| VEC | 4 | 4 | 5 | 5 | 1 | 4.1 |

### Example 5: Estimation of Microhardness and Elastic Modulus

The microhardness and modulus of elasticity of the alloys were measured using an Anton Par microhardness tester equipped with a V-M 53 Vickers indenter and a diamond tip. A 100 mN force was applied at a controlled rate of 200 mN/min with a 5 second hold at peak load. Nine indentations were spaced 0.5 mm apart to ensure representative measurements. The Oliver and Pharr technique was used to determine the hardness and Young's modulus, which included analyzing the loading and unloading curves for various parameters [Oliver, W. C. and Pharr, G. M., An improved technique for determining hardness and elastic modulus using load and displacement sensing indentation experiments, Journal of Materials Research, 1992, 7, 1564, which is incorporated herein by reference in its entirety]. The loading and unloading curves yielded parameters such as maximum load (Pₘₐₓ), maximum displacement (hₘₐₓ), final depth after indenter removal (h_{f}), and contact stiffness (S), which is defined as the slope of the upper portion of the unloading curve during the initial stages of unloading. The Aₚ for a Vickers indenter with a face angle of 68° was calculated using the parabolic equation 6.

$${A}_{p} = 24.5 {h}^{2} + 4163 h \qquad ( Eq . 6 )$$

After measuring the Aₚ, the hardness was estimated from equation 7.

$$Hardness = \frac{{P}_{max}}{{A}_{p}} \qquad ( Eq . 7 )$$

The elastic modulus, on the other hand, was measured by relating the Aₚ to contact stiffness through equation 8.

$$S = β \frac{2}{\sqrt{π}} {E}_{ind} \sqrt{{A}_{p}} \qquad ( Eq . 8 )$$

Equation 9 defines E_{ind} as the effective indentation modulus, while R is a dimensionless quantity related to indenter geometry.

$$\frac{1}{{E}_{ind}} = \frac{1 - {v}^{2}}{E} + \frac{1 - {v}_{i}^{2}}{{E}_{i}} \qquad ( Eq . 9 )$$

vᵢ is the Poisson's ratio of the indenter, Ei is the elastic modulus of the indenter, E is the elastic modulus of the sample and v is the Poisson's ratio of the sample.

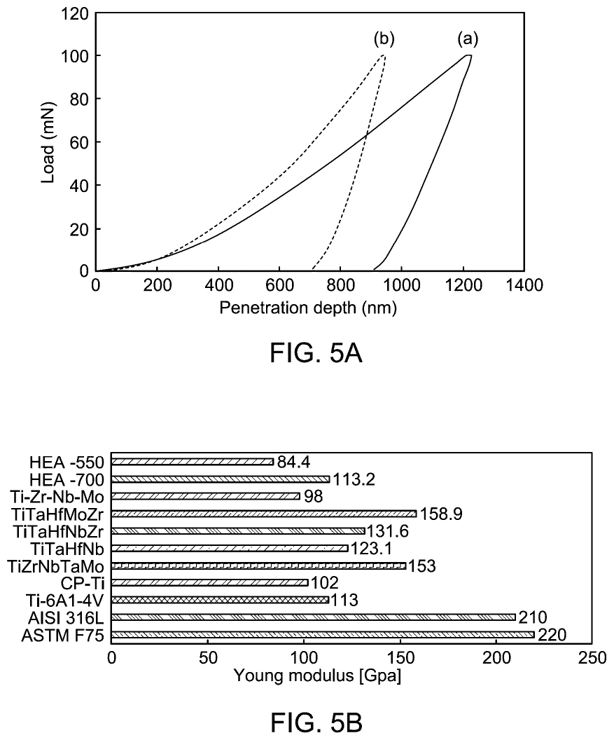

HEA's mechanical properties were assessed using a micro indentation test [Diez-Perez, A. et al., Microindentation for In Vivo Measurement of Bone Tissue Mechanical Properties in Humans, J Bone Miner Res., 2010, 25(8). 1877-1885, which is incorporated herein by reference in its entirety]. The loading and unloading curves obtained during the micro indentation testing are shown in FIG. 5A (a and b). The hardness and Young's modulus were estimated according to Oliver and Pharr [Oliver, W. C. and Pharr, G. M., An improved technique for determining hardness and elastic modulus using load and displacement sensing indentation experiments, Journal of Materials Research, 1992, 7, 1564, which is incorporated herein by reference in its entirety]. The HEA's Vickers microhardness (HV) was found to be 3.47±0.1 GPa and 5.35 GPa for samples compacted at 550 and 700 MPa, respectively. The improved microhardness of the HEA 700 MPa is related to the improvement in densification caused by increasing the compaction pressure, as the measured density of samples compacted at 700 MPa was 6.508 g/cm³, compared to 5.874 g/cm³ for samples compacted at 550 MPa. HEA microhardness does not just follow the value calculated using the “rule of mixtures” and the constituent elements′ characteristics. Table 1 lists each component's HV values at room temperature. Based on the law of mixtures, the HEA microhardness of mixtures was found to be (HV)ₘᵢₓ=0.975 GPa. The hardening effect observed in the developed HEA samples may be attributed to the solid solution-like strengthening [Wang, S-P. et al., (TiZrNbTa)—Mo high-entropy alloys: Dependence of microstructure and mechanical properties on Mo concentration and modeling of solid solution strengthening, Internietallics, 2018, 95, 59, which is incorporated herein by reference in its entirety]. The HEA exhibited an elastic modulus of 84.4 GPa and 113.2 GPa for samples compacted at 550 and 700 MPa, respectively (FIG. 5B). FIG. 5B displays the different biomedical alloys′ elastic modules in comparison to the metallic biomaterials and developed HEA. HEA compacted at 550 MPa exhibited a reduction in the modulus with 84.4±8.7 GPa, when compared to Ti64 alloy, CoCrMo alloy, and stainless alloys. The modulus of bone is 10 to 40 GPa, and HEA compacted at 550 MPa exhibits a modulus close to natural bone when compared to Ti64 alloy, CoCrMo, and stainless alloy counterparts [Walunj, (G. et al., Light weight-low modulus biocompatible titanium alloys processed via spark plasma sintering, Journal of Alloys and Metallurgical Systems, 2023, 3, 100018; and Liang, S., Review of the design of titanium alloys with low elastic modulus as implant materials, Adv. Eng. Mater., 2020, 22, 2000555, both of which are incorporated herein by references in their entireties]. Designing bio-implants is a careful balance between mechanical properties and biocompatibility to prevent/reduce stress shielding at the bone-implant interface. The mechanical biocompatibility of Ti alloys influences their suitability as implant materials. The “stress shielding” effect, which arises from shear stresses due to the difference of material properties between bone and implant, is a prevalent problem among mechanical biocompatibilities [Chmielewska, A. and Dean, D., The role of stiffness-matching in avoiding stress shielding-induced bone loss and stress concentration-induced skeletal reconstruction device failure, Acta Biomaterialia, 2023, 173, 51, which is incorporated herein by reference in its entirety]. A reason for the “stress shielding” phenomenon is the disparity in elastic modulus between the implants and the natural bone. Because of its higher elastic modulus than bone, the implant material is subjected to large forces from the body. The bone, in a state of relaxation, lacks sufficient mechanical strain to maintain its strength, density, and structural integrity, which may result in bone resorption, implant loosening, and early failure. The modulus of a Ti alloy is determined by a complex interplay of phases, crystal structure, composition, and bonding forces [Hussein, M. A. et al., Design, processing, and biocorrosion study of Ti-30Nb alloy with low elastic modulus for bioimplant applications, Mater. Chem. Phys., 2023, 309, 128413, which is incorporated herein by reference in its entirety]. This varying modulus within the alloy can be traced back to its microscopic structure. Mechanical compatibility of high load-bearing implants is carefully considered. While α+β Ti alloys are less stiff than cobalt and iron-based biomedical alloys, they are still stiffer than human bone. [Rho, J. Y. et al., Young's modulus of trabecular and cortical bone material: ultrasonic and microtensile measurements, J. Biomech., 1993, 26, 111, which is incorporated herein by reference in its entirety]. The HEA of the current disclosure showed a lower modulus value compared to previously developed alloys of Ti—Zr—Nb—Ta—Mo, Ti—Ta—Hf—Nb—Zr, and Ti—Zr—Nb—Mo HEA [Wang et al., TiZrNbTaMo high-entropy alloy designed for orthopedic implants: As-cast microstructure and mechanical properties, Mater. Sci. Eng:C, 2017,73; Gurel et al., Fracture behavior of novel biomedical Ti-based high entropy alloys under impact loading, Mater. Sci. Eng: A, 2021, 803, 140456; Wong et al., Structure and properties of Ti-rich Ti—Zr—Nb—Mo medium-entropy alloys, J. Alloys Compd 2021, 868, 159137, each of which are incorporated herein by reference in their entirety]. Alloys that possess a high elastic modulus are prone to inducing “stress shielding” and are thus unsuitable for long-term implantation as a replacement for hard tissue. Avoidance and prevention of the “stress shielding” phenomenon is beneficial for the proper installation of bio-implants. The HEA alloy of the current disclosure is in the preferred range as an implant [Gaur, P. et al., Friction and wear properties of biocomposites for dental, orthopaedic, and biomedical applications, Tribiological Properties, Performance and Applications of Biocomposites, 2024, 12, 185-218, which is incorporated herein by reference in its entirety].

### Example 6: Corrosion Analysis of High Entropy Alloy (HEA)

In vitro corrosion studies of the HEA of the current disclosure and commercial biomedical Ti64 alloy were assessed in simulated body fluid (SBF). Gamry potentiostat (Model: Reference 3000, USA) was utilized through a three-electrode cell set-up containing SBF as an electrolytic medium. Bare Ti64 and HEA alloys with a surface area of 1.76 cm² were represented as working electrodes, whereas graphite rod and saturated calomel electrode (SCE) were respectively used as auxiliary and reference electrodes. Before performing the corrosion analyses, the monitoring of open circuit potential (OCP) was done for about 1800 seconds to accomplish steady equilibrium conditions. Potentiodynamic polarization (PDP) measurements were performed to measure the alloy's corrosion performance on the investigated HEA samples by selecting the potential range of +0.250 V vs. OCP and 2.00 V vs SCE using a scanning rate of 1 mV/s. Electrochemical impedance spectroscopic (EIS) analysis was performed on HEA samples in the frequency range of 100 kHz to 10 mHz through a 10 mV amplitude. To analyze the obtained EIS data, the EIS simulation procedure was completed using the Echem Analyst, which allowed observing the Chi-square (χ²) value to inspect the eminence of the simulation analyses. All corrosion tests were reiterated at least three times and are reproducible.

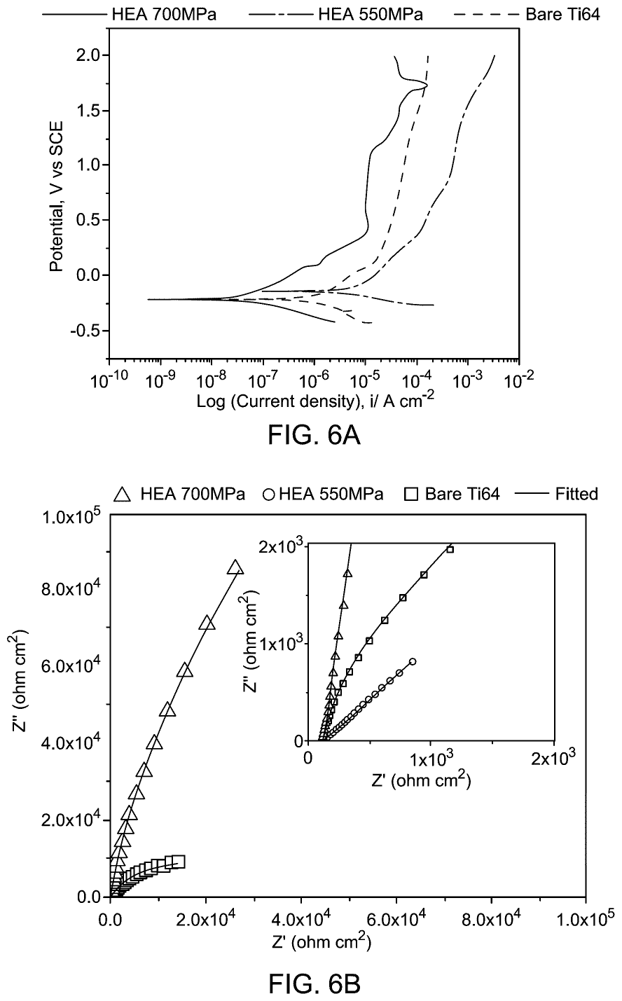

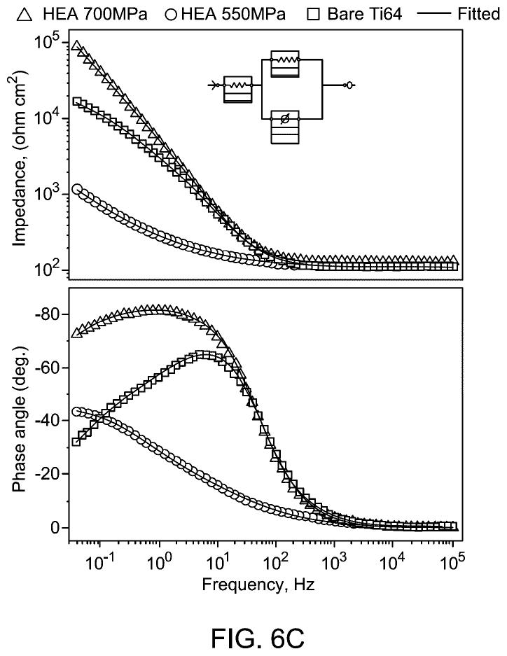

The corrosion results tested in SBF of HEA compacted at 550 MPa and 700 MPa compared to Ti64 alloy are shown in FIGS. 6A-6C, and the extracted values from the PDP curves are summarized in Table 2, consisting of corrosion current density (i_{corr}), corrosion potential (E_{corr}), and passive current density (iₚₐₛₛ). The PDP plot (FIG. 6A) of the HEA700 sample shows different anodic branches compared with the HEA550 and Ti64 alloy substrates. The vertical anodic branch of the HEA700 substrate represents reduced anodic metal dissolution [Hussein, M. A. et al., Ti-30Nb-3Ag alloy with improved corrosion resistance and antibacterial properties for orthopedic and dental applications produced by mechanical alloying, Journal of the J. Mech. Behav. Biomed. Mater., 2023, 142, 105851, which is incorporated herein by reference in its entirety]. Unlike the Ti64 alloy, the HEA700 alloy displays a decreased current density in the segment ranging from the beginning of an anodic potential to around 2.0 V. HEA700 substrates display a passivation region in their anodic segment, supporting a passive e-film formation in the tested medium. The iₚₐₛₛ value of the HEA700 alloy was about two orders of magnitude less than that of Ti64 in the SBF medium, suggesting a reduction in the dissolution of a passive film on the HEA700 substrate [Bai, Y. et al., Improved corrosion behaviour of electron beam melted Ti-6A1-4V alloy in phosphate buffered saline, Corrosion Sci., 2017, 123, 289, which is incorporated herein by reference in its entirety].

EIS results for the HEA and Ti64 alloys in the SBF medium are presented in FIG. 6B (Nyquist plot) and FIG. 6C (Bode diagram). A distorted semi-circle arc with linear-like performance (FIG. 6B) was exhibited by all the examined samples, demonstrating that the passivation layer of the Ti alloy surface disrupts the charge transfer reactions at the interface of Ti/SBF. Further, the larger diameter of the semicircle arc suggests that there is more resistance to the charge transfer reaction at the metal-electrolyte interface [Zhou et al., Microstructure, corrosion behavior and cytotoxicity of Zr—Nb alloys for biomedical application, Mater. Sci. Eng. C 2012, 32, 851, which is incorporated herein by reference in its entirety]. In comparison to the HEA500 and Ti64 samples, the capacitance arc diameter in the SBF medium was larger in the HEA700 sample.

In the Bode diagram, as shown in FIG. 6C, due to the single relaxation process, the tested samples showed one time constant, indicating the existence of a comparable electrochemical process on the Ti substrate in SBF [Subramanian, R. et al., Structural and tribological properties of DC reactive magnetron sputtered titanium/titanium nitride (Ti/TiN) multilayered coatings, Surf Coat. Technol., 2011, 205, 3485, which is incorporated herein by reference in its entirety]. Within the lower frequency segment, the HEA700 alloy's impedance modulus revealed the highest impedance value. Specifically, the phase angle was measured at approximately −80° and the impedance at the low-frequency region was detected on the order of 10⁵ Ω cm² in the SBF medium. Higher impedance and phase angle values up to −90° are ascribed to the materials' capacitive characteristics, which result in enhanced corrosion resistance because of the passive layer on the Ti substrate. Thus, the EIS data suggest that the HEA700 substrate showed effective surface protective performance in the SBF medium.

The measured EIS data are further inspected by choosing different equivalent circuit models (ECMs) consisting of a series of electrical circuit parameters, illustrating the electrochemical phenomenon happening at the interface of the bare Ti surface and SBF medium. The gained EIS curves can be fitted by one-time constant equivalent circuit Rₛ(R_{ct}CPE_{dl}) (FIG. 6C, inset), where Rₛ and R_{ct} represent the solutions and the charge transfer resistances, respectively, and CPE represents a constant phase element (CPE), accounting to the capacitance performance behavior of the Ti surface [Madhan Kumar, A. et al., Preparation and characterization of pectin/polypyrrole based multifunctional coatings on TiNbZr alloy for orthopaedic applications, Carbohydr. Polym., 2020, 242, 116285; and Hussein, M. A. et al., Laser nitriding of the newly developed Ti-20Nb-13Zr at. % biomaterial alloy to enhance its mechanical and corrosion properties in simulated body fluid, J. Mater. Eng. Perform., 2017, 26, 5553, both of which are incorporated herein by references in their entireties]. From Table 2, the values of Rₑₜ for the HEA700 specimen were higher than those of the HEA550 and Ti64 specimens, demonstrating that the corrosion reaction was competently hindered by the Ti/SBF interface. Moreover, the low CPE_{dl} values of the HEA700 revealed minor dissolution and improved the stability of the passive film. Among the investigated samples, HEA550 showed the lowest resistance against corrosion in the SBF medium, which may be due to inadequate compaction and higher porosity after the compaction. The sample compacted at 550 MPa posed a relative density of 85.25%, while the sample sintered at 700 MPa showed a 94.45% relative density. The high porosity of HEA samples compacted at 550 MPa (14.75%) may trap ionic species and hinder the formation of uniform passive film. The data were attributed to the effective stability of the passive layer formed on HEA sintered at 1300° C. and compacted at 700 MPa that protects the Ti surface by obstructing the attack of hostile species from the SBF medium.

TABLE 2
Electrochemical corrosion parameters of HEA in SBF medium

| | E_{corr} | i_{corr} | iₚ | R_{ct} | Q_{dl} | |
|---|---|---|---|---|---|---|
| Sample | mV | μA cm⁻² | μA cm⁻² | Ω cm² | μΩ⁻¹ · cm⁻² · sⁿ | n_{dl} |
| Ti64 | −0.186 | 1.187 | 45.215 | 16924.25 | 25.658 | 0.972 |
| HEA550MPa | −0.125 | 13.078 | 537.684 | 1196.54 | 112.358 | 0.945 |
| HEA700MPa | −0.182 | 0.068 | 9.325 | 91778.35 | 1.259 | 0.991 |

A non-equiatomic 35Ti-35Zr-20Nb-5Ta-5Ag HEA was designed and processed via powder metallurgy with a potential for bioimplant applications. The nanocrystalline bcc HEA alloyed powder was obtained with a crystallite size of 3.6 nm after 20 hours of mechanical alloying. The sintered HEAs exhibit a dual-phase bcc structure and an ultrafine Zr-rich equiaxed phase with grain sizes of 1.5 μm and 1.8 μm for HEA compacted at 550 and 700 MPa, respectively. Further, the HEA had a lower modulus of 84.4 GPa and 113.2 GPa compacted at 550 and 700 MPa, respectively, compared to Ti64 alloys, biomedical grades of Co-based alloys, and stainless steels. HEA compacted at 700 MPa showed higher impedances and reduced the relevant corrosion current densities compared to that of the HEA550 MPa and commercial Ti64 alloy, corroborating the efficient passive protection characteristics of the HEA700 MPa in the SBF medium. Ti—Zr—Nb—Ta—Ag HEA, with low elastic modulus and improved corrosion properties in simulated body fluid, is a promising bioimplant candidate.

Numerous modifications and variations of the present disclosure are possible in light of the above teachings. It is therefore to be understood that within the scope of the appended claims, the disclosure may be practiced otherwise than as specifically described herein.

1: A non-equiatomic high entropy alloy, comprising:
titanium in an amount of 33 to 37 atomic percent;
zirconium in an amount of 33 to 37 atomic percent;
niobium in an amount of 18 to 22 atomic percent;
tantalum in an amount of 3 to 7 atomic percent; and
silver in an amount of 3 to 7 atomic percent;
wherein atomic percent is based on the total atom count of the non-equiatomic high entropy alloy,
wherein the titanium, zirconium, niobium, tantalum, and silver are randomly distributed within the non-equiatomic high entropy alloy.

2: The non-equiatomic high entropy alloy of claim 1, wherein the alloy is made by a process comprising:
mixing a titanium powder, a zirconium powder, a niobium powder, a tantalum powder, and a silver powder to form a first mixture;
ball-milling the first mixture for 16 to 24 hours at a speed of 250 to 350 rpm;
pressing the first mixture at a pressure of 500 to 750 MPa; and
sintering the first mixture to a temperature of 1250 to 1350° C. to form the alloy.

3: The non-equiatomic high entropy alloy of claim 2, wherein the ball-milling has a bypass ratio of 5:1 to 15:1 in an inert environment.

4: The non-equiatomic high entropy alloy of claim 2, wherein, after the pressing, the sintering includes heating the first mixture to a temperature of 1250 to 1350° C. at a rate of 8 to 12° C./min to form the alloy.

5: The non-equiatomic high entropy alloy of claim 2, wherein the sintering occurs for 1 to 3 hours.

6: The non-equiatomic high entropy alloy of claim 1, wherein the alloy has a valence electron concentration of 3 to 5.

7: The non-equiatomic high entropy alloy of claim 1, wherein the alloy has an atomic-size difference (6) of 4 to 6%.

8: The non-equiatomic high entropy alloy of claim 1, wherein the alloy has an omega (Q) parameter of 10 to 11.

9: The non-equiatomic high entropy alloy of claim 1, wherein the alloy comprises a major body-centered cubic 1 (bcc1) phase, a grain boundary body-centered cubic 2 (bcc2) phase, and an ultra-fine zirconium-rich equiaxed phase.

10: The non-equiatomic high entropy alloy of claim 1, wherein the alloy has a grain size of 1 to 2 μm.

11: The non-equiatomic high entropy alloy of claim 1, wherein the alloy has a crystallite size of 3 to 4 nm.

12: The non-equiatomic high entropy alloy of claim 1, wherein the alloy has a melting temperature of 2150 to 2250 K.

13: The non-equiatomic high entropy alloy of claim 1, wherein the alloy has a density of 6.5 to 7.5 g/cm³.

14: The non-equiatomic high entropy alloy of claim 1, wherein the alloy has a Vickers microhardness of 3 to 6 GPa.

15: The non-equiatomic high entropy alloy of claim 1, wherein the alloy has an elastic modulus of 80 to 120 GPa.

16: The non-equiatomic high entropy alloy of claim 1, wherein a rate of antibacterial inhibition of the alloy in the presence of Bacillus subtilis is 90 to 94% greater compared a rate of antibacterial inhibition of a commercially pure titanium sample.

17: The non-equiatomic high entropy alloy of claim 1, wherein a rate of antibacterial inhibition of the alloy in the presence of Escherichia coli is 83 to 90% greater compared a rate of antibacterial inhibition of a commercially pure titanium sample.

18: The non-equiatomic high entropy alloy of claim 1, wherein the alloy has an impedance value of 70,000 to 300,000 Ω cm² in a stimulate body fluid medium.

19: The non-equiatomic high entropy alloy of claim 1, wherein the alloy has a phase angle of −75° to −85° in a stimulate body fluid medium.

20: A bioimplant material, comprising:
the non-equiatomic high entropy alloy of claim 1.
